(19) United States
(12) Patent Application Publication
(10) Pub. No.: US 2016/0296312 A1
(43) Pub. Date: Oct. 13, 2016

(54) CHRONIC CRANIAL WINDOW ALLOWING DRUG APPLICATION, CELLULAR MANIPULATIONS, AND ELECTROPHYSIOLOGY

(71) Applicant: OKINAWA INSTITUTE OF SCIENCE AND TECHNOLOGY SCHOOL CORPORATION, Okinawa (JP)

(72) Inventors: Bernd KUHN, Okinawa (JP); Christopher Joel ROOME, Okinawa (JP)

(73) Assignee: Okinawa Institute of Science and Technology School Corporation, Okinawa (JP)

(21) Appl. No.: 15/035,825

(22) PCT Filed: Dec. 16, 2014
(86) PCT No.: PCT/JP2014/006262

§ 371 Date: May 11, 2016

Related U.S. Application Data
(60) Provisional application No. 61/918,193, filed on Dec. 19, 2013.

(51) Int. Cl. A61D 7/00; A61B 46/20; A61F 13/12; A61B 5/00

(52) U.S. Cl. CPC A61D 7/00; A61B 5/6814; A61B 5/0042; A61B 46/20; A61F 13/12; A61B 2503/40; A61B 2503/42

(57) ABSTRACT

A cranial window with an accessing port for medical research includes a sheet-shaped member configured to be installed as a cranial window on an outer brain skin of an animal subject through an opening in a skull, the sheet-shaped member having an optically transparent window therein or in entirety thereof to allow optical imaging into a brain of the animal subject; and an access port in the sheet-shaped member for allowing sterile insertion and removal of an accessing member having a sharp tip, the access port being configured to be self-sealing when the accessing member is removed.

The present invention relates to research in neuroscience and pharmaceutical drug testing for neurological disorders in vivo. It permits long-term optical imaging combined with multiple targeted brain manipulations. This application hereby incorporates by reference U.S. Provisional Application No. 61/918,193, filed Dec. 19, 2013, and an article by C. J. Roome and B. Kuhn, entitled “Chronic Cranial Window with Access Port for Repeated Cellular manipulations, drug application, and electrophysiology,” Frontiers in Cellular Neuroscience, Vol. 8, November 2014, listed as Non-Patent Literature No. 5 below.

### TECHNICAL FIELD

### Background Art

In neuroscience and pharmaceutical drug research, versatile and cost-effective techniques for studying neurons and neuronal activities in awake animals over a long period of time have been long sought. In particular, a reliable and economical technique for studying brain diseases on a cellular level in vivo and evaluating the effects of drugs on brain diseases has been long desired.

For example, two-photon microscopy through a chronic cranial window for mice has been performed. See Non-Patent Literature No. 1. However, with that technique, although the brain is kept sterile, the brain region of interest is not accessible for local drug application or electrophysiology. Various different types of windows for imaging and acute drug/dye application have been also developed. See Non-Patent Literature No. 3. Furthermore, for large animals, like monkey and rats, very complex, sophisticated cranial window techniques are currently available. See Non-Patent Literature No. 4.

### CITATION LIST

### Non Patent Literature

NPL 1: “Long-term, high-resolution imaging in the mouse neocortex through a chronic cranial window”: A. Holtmaat, et al. (2009) Nature Protocols 4, 1128 -1144
NPL 2: M. J. Lopez-Martinez and E. M. Campo (2011). “Micro-Nano Technologies for Cell Manipulation and Subcellular Monitoring, Biomedical Engineering”—From Theory to Applications, Prof. Reza Fazel (Ed.), ISBN: 978-953-307-637-9, InTech, Available from:
http://www.intechopen.com/books/biomedical-engineering-from-theory-toapplications/micro-nano-technologies-for-cell-manipulation-and-subcellular-monitoring

### NPL 3: F. Helmchen & W. Denk (2005), Nature Methods 2, 932-940

NPL 4: A. Arieli et al. (2002) J. Neurosci. Meth. 114, 119 &#8211; 133
NPL 5: C. J. Roome and B. Kuhn, “Chronic Cranial Window with Access Port for Repeated Cellular manipulations, drug application, and electrophysiology,” Frontiers in Cellular Neuroscience, Vol. 8, November 2014

### SUMMARY OF INVENTION

### Technical Problem

In the system described in NPL 4, the windows are made of several parts and require time consuming surgeries with an increased danger of infections. Large animals are, however, not necessarily useful for many research projects, and drug searches because they are very expensive and very difficult to deal with. But these complex sophisticated windows are not suited for small animals like mice.

Accordingly, the present invention is directed to research tools in neuroscience and pharmaceutical drug testing for neurological disorders in vivo.

An object of the present invention is to provide a system that permits long-term optical imaging combined with multiple targeted brain manipulations.

Another object of the present invention is to provide an inexpensive, simple, reliable and sterile window that allows access to the brain region of small animals, like mice, for imaging and manipulation.

### Solution to Problem

To achieve these and other advantages and in accordance with the purpose of the present invention, as embodied and broadly described, in one aspect, the present invention provides a cranial window with an access port for medical research or treatment, comprising: a sheet-shaped member configured to be installed as a cranial window on an outer brain skin of an animal subject through an opening in the skull, the sheet-shaped member having an optically transparent window therein or in entirety thereof to allow optical imaging into a brain of the animal subject; and an access port in the sheet-shaped member for allowing sterile insertion and removal of an accessing member having a sharp tip, the access port being configured to be self-sealing when the accessing member is removed.

In the above aspect, the access port may be configured such that at least the tip of the accessing member may be visible through the optically transparent window of the sheet-shaped member when the accessing member is inserted.

In the above aspect, the access port may include a membrane made of transparent or opaque silicone, sealing an opening formed in the optically transparent window of the sheet-shaped member.

In the above aspect, the opening may be a round hole, a slit, or a half-ring shaped hole.

In the above aspect, the accessing member may be a pipette to inject a substance into the brain, or an electric probe.

In the above aspect, the access port may be located within the optically transparent window of the sheet-shaped member.

The cranial window of the above aspect of the present invention may further include one or more of additional said access ports.

The cranial window of the above aspect of the present invention may further include one or more of additional said access ports each comprising a membrane made of transparent or opaque silicone, sealing an opening formed the optically transparent window of the sheet-shaped member.

The cranial window of the above aspect of the present invention may further include an electronic component embedded or installed on the sheet-shaped member. The electronic component may include a bath electrode.

In another aspect, the present invention provides a cranial cover sheet for medical research or treatment, comprising: a sheet-shaped member configured to be installed over an outer brain skin of an animal subject through an opening in the skull; and an access port in the sheet-shaped member for allowing sterile insertion and removal of an accessing member having a sharp tip, the access port being configured to be self-sealing when the accessing member is removed.

In the above aspect, the access port may include a membrane made of transparent or opaque silicone, sealing an opening formed in the sheet-shaped member.

The cranial cover member of the above aspect may further include an electronic component embedded or installed on the sheet-shaped member. The electronic component may include a bath electrode.

### Advantageous Effects of Invention

According to one or more aspects of the present invention, in-vivo brain manipulations can be performed repeatedly and in combination with optical imaging, and these experiments are now easily repeated in a single animal over a longer time period in a very cost-effective manner. Various aspects of the present invention can significantly simplify current experiments and, even more importantly, make it possible to perform many new experiments related to in vivo drug screening, which were not possible before. The technique is specifically useful for small animals like mice where imaging is done through the dura which acts as an additional and even re-growing seal of the brain. Because of the biocompatibility of the window, the animals recover quickly from the surgery and can be used for weeks or months (likely a year or longer). With this technique, the number of animals used for research can be significantly reduced while the information gained from a single animal can be dramatically increased. For example, a time course of local drug application to the brain over a few months can be performed in a single animal while the effects of the drug can be repeatedly monitored during such a period. Before this invention, many animals would have been sacrificed at different times to achieve similar results. The shape and size of the glass window as well as the number and the shape and size of the accessing port (a silicone membrane as an example) can be determined appropriately to meet the specific needs of the experiments or treatments.

Additional or separate features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

It is to be understood that both the foregoing general description and the following detailed description are exemplary and explanatory, and are intended to provide further explanation of the invention as claimed.

### BRIEF DESCRIPTION OF DRAWINGS

FIG. 1 shows a schematic plan view (left) and a schematic cross-sectional view (right) of a glass window according to an embodiment of the present invention.

FIG. 2 is a miscroscopic image taken through a cranial window of an embodiment of the present invention, installed on the outer brain skin of a mouse after a craniotomy is performed.

FIG. 3 shows that a glass piece is clamped by a crocodile clip with silicon tubing covering the teeth.

FIG. 4 shows the glass pieces of FIG. 3 after drilling

FIG. 5 shows the glass pieces of FIG. 3 with a silicone membrane

FIG. 6 shows a craniotomy of a deeply anesthetized mouse. The dura mater is exposed, but the brain is not.

FIG. 7 shows that the window of an embodiment of the present invention is mounted on the dura mater and sealed with a super glue to the bone. This allows chronic cranial imaging and simultaneous access to the brain through the membrane.

FIG. 8 shows that a headplate is installed to the skull with dental acrylic, thereby completing the surgery.

FIG. 9 shows an exemplary application of the cranial window of an embodiment of the present invention to an in-vivo experiment in which the brain activity is being imaged in an awake, head-fixed mouse on a spherical treadmill. With a micropipette, drugs could be injected into the brain through the silicone membrane. Or with an electrode, electrical signals can be recorded through the silicone membrane.

FIG. 10 is a side view drawing of the arrangement of FIG. 9 with additional details together with a plan view of the metal headplate shown on the upper side.

FIG. 11 is a side view drawing of the arrangement of FIG. 9 with an additional silicone membrane with a microelectrode for recoding or other purposes.

### DESCRIPTION OF EMBODIMENTS

A 5 mm diameter glass cranial window has been commonly used for in-vivo optical imaging in mice. In the conventional technique, however, after a craniotomy, the window re-seals the mouse skull, and any brain manipulations must be performed either during the initial surgery or subsequently by removing and reattaching the cranial window. This method is difficult and ultimately limits the number of possible manipulations (only one or two) during the time period over which such experiments can be performed on a single animal. See NPL documents 1 to 3, for example.

In an embodiment of the present invention, a 5 mm diameter glass coverslip (thickness 170 micron) was used, and a 1.5 mm hole was drilled through the glass using a diamond drill bit or a cone-shaped polishing stone drill bit. The hole was then sealed with a silicone glue (transparent or opaque) to form an air-tight and biocompatible “membrane.”

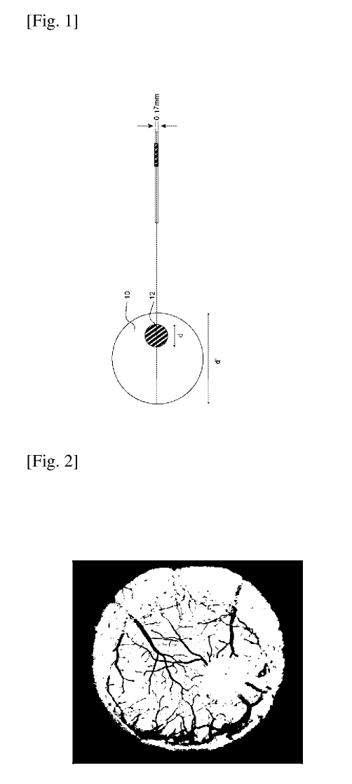

FIG. 1 shows a schematic plan view (left) and a schematic cross-sectional view (right) of a glass window according to an embodiment of the present invention. As shown in the figure, a glass window 10 (coverslip) has an opening filled with a silicone membrane 12. In this example, the diameter of the glass window is 5 mm, and the diameter of the silicone membrane is 1 mm

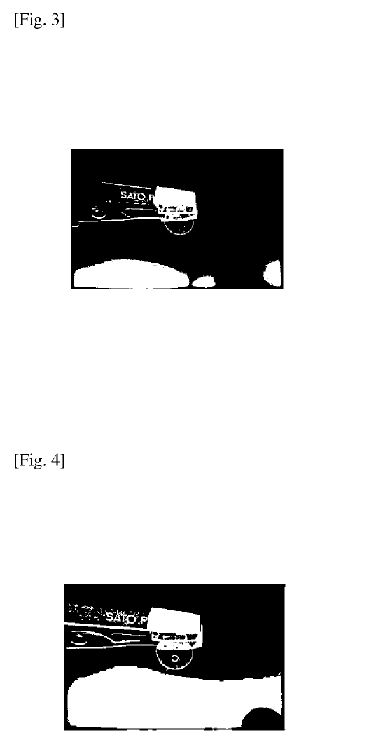

After the silicone glue has set, the glass can be sterilized and stored until it is used for surgery. After a craniotomy is performed, this modified glass window can be directly mounted on the dura (outer brain skin) and sealed with a super glue to the skull. The window hole filled with silicone now permits repetitive and targeted access to the brain for application of drugs or other compounds via glass or quartz micropipette. FIG. 2 is a miscroscopic image taken through a cranial window of the embodiment of the present invention, installed on the outer brain skin of a mouse after the craniotomy is performed. As shown in the figure, the access port is readily available within the observation window in this example.

The silicone membrane re-seals after the pipette for drug/compound application is removed from the brain. Therefore, the silicone membrane can maintain the sterile condition in the brain. The sterile condition and the lowest possible immune response are required for optimal optical imaging and testing of drugs.

The most significant advantage of this invention is that in-vivo brain manipulations can be performed repeatedly in combination with optical imaging, and these experiments are now easily repeated in a single animal over a longer time period. Various aspects of the present invention can significantly simplify current experiments and, even more importantly, make it possible to perform many new experiments related to in vivo drug screening, which were not possible before. The technique is specifically useful for small animals like mice where imaging is done through the dura which acts as an additional and even re-growing seal of the brain. Because of the biocompatibility of the window, the animals recover quickly from the surgery and can be used for weeks or months (likely a year or longer). With this technique, the number of animals used for research can be significantly reduced while the information gained from a single animal can be dramatically increased. For example, a time course of local drug application to the brain over a few months can be performed in a single animal while the effects of the drug can be repeatedly monitored during such a period. Before this invention, many animals would have been sacrificed at different times to achieve similar results. The shape and size of the glass window as well as the number and the shape and size of the accessing port (a silicone membrane as an example) can be determined appropriately to meet the specific needs of the experiments or treatments.

Main targets of imaging and manipulations that can be achieved by the present invention are neurons, glia, and brain vasculature. Furthermore, a bath electrode or other sensing devices, or like electronic components may be permanently integrated into the window of the present invention by known methods and techniques.

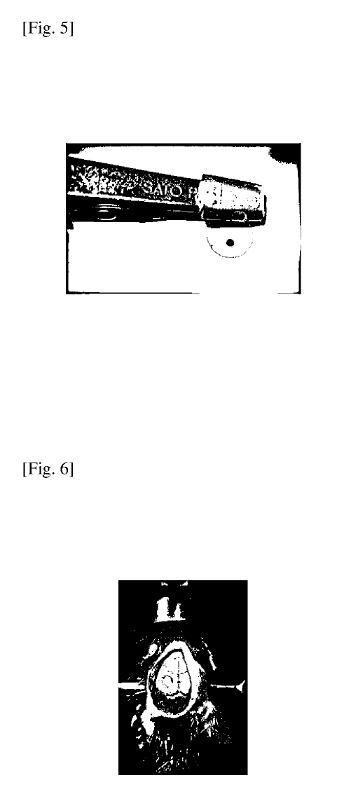

A glass window with an access port according to an embodiment of the present invention can be made by the following manner, which has been developed by the preset inventors. A 5 mm diameter glass coverslip (window) is first secured for drilling This can be achieved by clamping the glass coverslip using an electrical crocodile clip with teeth projected by silicone tubing to prevent breaking the glass (FIG. 3). Once the window is secure, a commercially available diamond tip drill bit (0.9 mm diam., for example) or cone-shaped polishing stone drill bit can be used to drill a 1.5 mm hole through the glass coverslip (FIG. 4). After drilling the hole through the glass coverslip, a drop of silicone glue (such as Kwik-Cast (Trademark) (opaque) or Kwik-Sil (Trademark) (transparent), both marketed by World Precision Instruments, Inc.) is applied to the center of the hole using a sharp object, such as an old drill bit (or toothpick), and the glue is allowed to contact only the edges of the hole to thereby form a silicone membrane with an airtight seal with the glass (FIG. 5). After the silicone glue has set (approx. 10 mins), the window can be sterilized and then used in a craniotomy.

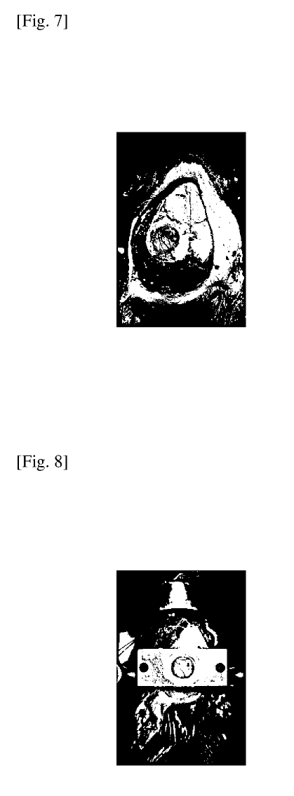

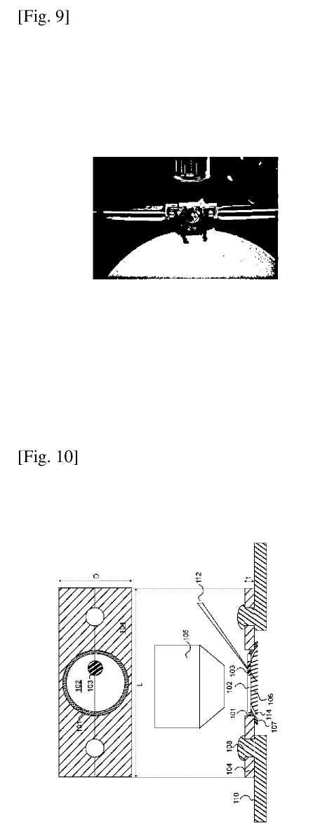

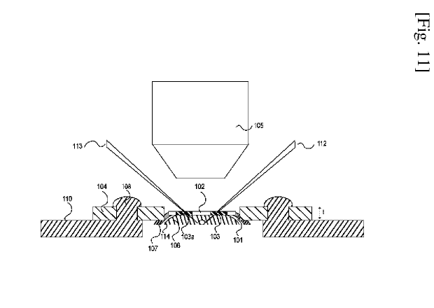

FIG. 6 shows a craniotomy of a deeply anesthetized mouse. The dura mater is exposed. During the craniotomy, the skin covering the skull is cut and a circular segment (approx. 3 mm in diameter) of the skull covering the brain is removed (FIG. 6) by drilling with a dental drill, for example. The glass window with a silicone membrane according to an embodiment of the present invention is placed over the exposed brain and fixed to the skull along the outside edge of the window using a super glue (101 in FIG. 10 below), forming a sterile and airtight seal over the brain. FIG. 7 shows that the window of an embodiment of the present invention is mounted on the dura mater and sealed with the super glue to the bone. This allows chronic cranial imaging and simultaneous access to the brain through the membrane.

As shown in FIG. 8, a metal headplate (20 mm in length, 8 mm in width, 1 mm in thickness t) is then placed over the window and attached to the underlying skull (114 in FIG. 10 below) using membrane dental acrylic (107 in FIG. 10 below) using screws 108 (FIG. 10), completing the surgery.

FIG. 9 shows an exemplary application of the cranial window of an embodiment of the present invention to an in-vivo experiment in which the brain activity is being imaged in an awake, head-fixed mouse on a spherical treadmill. With a micropipette, drugs could be injected into the brain through the silicone membrane. Or with an electrode, electrical signals can be recorded through the silicone membrane.

FIG. 10 is a side view drawing of the arrangement of FIG. 9 with additional details together with a plan view of the metal headplate shown on the upper side. FIG. 11 is a side view drawing of the arrangement of FIG. 9 with an additional silicone membrane with a microelectrode for recoding or other purposes. During the experiment, the metal headplate 104 is used to secure the mouse to the microscope stage 110 (FIG. 10). A microscope objective lens 105 is positioned above the glass window 102 for neuro-optical imaging into the brain 106 of the mouse. A quartz (or borosilicate) glass pipette 112 can be inserted through the silicone membrane 103 and into the brain 106 to inject a drug, virus or fluorescent dye for neuronal labeling. When the injection is completed, the pipette 112 can be retracted allowing the silicone membrane 103 to reseal and maintain the sterile seal to the brain 106. Targeted single cell drug or fluorescent dye injection can be performed with simultaneous optical imaging. Alternatively, a microelectrode 112 (or 113) can be inserted through the silicone membrane 103 into the brain 106 for single cell electrical recording, or used in combination with local drug application by including a second window hole (with silicone membrane) 103a during the window fabrication, as shown in FIG. 11. This window is also useful for small animals like mice or zebra fish.

It will be apparent to those skilled in the art that various modification and variations can be made in the present invention without departing from the spirit or scope of the invention. Thus, it is intended that the present invention cover modifications and variations that come within the scope of the appended claims and their equivalents. In particular, it is explicitly contemplated that any part or whole of any two or more of the embodiments and their modifications described above can be combined and regarded within the scope of the present invention.

### REFERENCE SIGNS LIST

10, 102 Glass Window
12, 103 Silicone Membrane
101 Super Glue
104 Metal Headplate
105 Microscope Objective
106 Brain of mouse
107 Dental Acrylic
108 Screw
110 Microscope Stage
112 Injection Pipette or Microelectrode
113 Microelectrode

1. A cranial window with an accessing port for medical research or treatment, comprising:
a sheet-shaped member configured to be installed as a cranial window on an outer brain skin of an animal subject through an opening in a skull, the sheet-shaped member having an optically transparent window therein or in entirety thereof to allow optical imaging into a brain of the animal subject; and
an access port in the sheet-shaped member for allowing sterile insertion and removal of an accessing member having a sharp tip, the access port being configured to be self-sealing when the accessing member is removed.

2. The cranial window according to claim 1, wherein the access port is configured such that at least the tip of the accessing member may be visible through the optically transparent window of the sheet-shaped member when the accessing member is inserted.

3. The cranial window according to claim 1, wherein the access port comprises a membrane made of transparent or opaque silicone, sealing an opening formed in the optically transparent window of the sheet-shaped member.

4. The cranial window according to claim 3, wherein the opening is a round hole.

5. The cranial window according to claim 3, wherein the opening is a slit.

6. The cranial window according to claim 3, wherein the opening is a half-ring shaped hole.

7. The cranial window according to claim 1, wherein the accessing member is a pipette to inject a substance into the brain.

8. The cranial window according to claim 1, wherein the accessing member is an electric probe.

9. The cranial window according to claim 1, wherein said access port is located within the optically transparent window of the sheet-shaped member.

10. The cranial window according to claim 1, further comprising one or more of additional said access ports.

11. The cranial window according to claim 1, further comprising one or more of additional said access ports each comprising a membrane made of transparent or opaque silicone, sealing an opening formed in the optically transparent window of the sheet-shaped member.

12. The cranial window according to claim 1, further comprising an electronic component embedded or installed on the sheet-shaped member.

13. The cranial window according to claim 12, wherein the electronic component includes an electrode.

14. A cranial cover sheet for medical research or treatment, comprising:
a sheet-shaped member configured to be installed over an outer brain skin of an animal subject through an opening in a skull; and
an access port in the sheet-shaped member for allowing sterile insertion and removal of an accessing member having a sharp tip, the access port being configured to be self-sealing when the accessing member is removed.

15. The cranial cover member according to claim 14, wherein the access port comprises a membrane made of transparent or opaque silicone, sealing an opening formed in the sheet-shaped member.

16. The cranial cover member according to claim 14, further comprising an electronic component embedded or installed on the sheet-shaped member.

17. The cranial cover member according to claim 15, wherein the electronic component includes a bath electrode.
